(19) United States
(12) Patent Application Publication
(10) Pub. No.: US 2018/0042327 A1
(43) Pub. Date: Feb. 15, 2018

(54) HARD HAT HAVING INTEGRAL MISTING SYSTEM

(71) Applicant: Jared Taylor, Mobile, AL (US)

(72) Inventor: Jared Taylor, Mobile, AL (US)

(21) Appl. No.: 15/232,765

(22) Filed: Aug. 9, 2016

(51) Int. Cl. A42B 3/04; A42B 3/14; A61F 7/10

(52) U.S. Cl. CPC A42B 3/048; A61F 7/10; A42B 3/14; A61F 2007/0068

(57) ABSTRACT

A hard hat having an integral misting system for directing a liquid from a stored reservoir onto a user's face and head as a cooling mist. The misting hard hat includes a hat base having a rigid shell and suspension system, a fluid reservoir attached to the interior of the rigid shell, a misting component for dispersing liquid as a mist, and a distribution system for delivering liquid from the from the reservoir to the misting component under pressure. In one embodiment, the misting component is formed from a clip on housing that includes a plurality of misting nozzles which each direct mist from a slightly different position and/or at a slightly different trajectory, thereby provided a large coverage area for mist.

### BACKGROUND OF THE INVENTION

### Field of the Invention

This invention relates generally to fluid dispersing apparel and, more particularly, to a hard hat that has an integrated, self contained misting system.

### Description of the Prior Art

The use and design of conventional hard hats used for protection of the head in workplace environments such as industrial or construction sites is well known. Hard hats generally aim to provide protection to a wearer from falling objects, impact with other objects, debris, rain, and electric shock and are often required by law or regulation for workers on such sites. A problem which still exists, however, arises from the use of hard hat in hot and/or humid weather because the wearing hat may make it more difficult for the user to maintain a comfortable and/or safe body temperature. Existing structural solutions to this problem, typically involving the use of integrated fans, generally only move around hot ambient air and thus may not make a substantial difference to a user. Thus, there remains a need for a hard hat having an integral misting system which would operate to cool a wearer by spraying mist on the wearer. It would be helpful if such a hard hat having an integral misting system included a reservoir allow for the storage of liquid to be misted. It would be additionally desirable for such a hard hat having a distribution system which included a pump and conduit network operative to deliver liquid in the reservoir to misting ducts.

The Applicant's invention described herein provides for a hard hat having an integral misting system adapted to allow a user to direct a liquid spray onto their neck and upper back area from the user's hard hat. The primary components in Applicant's hard hat having an integral misting system are a hat base, a reservoir, a distribution system and a misting component. When in operation, the hard hat having an integral misting system enables more effective maintenance of a safe body temperature. As a result, many of the limitations imposed by prior art structures are removed.

### SUMMARY OF THE INVENTION

A hard hat having an integral misting system for directing a liquid from a stored reservoir onto a user's neck and upper back area as a cooling mist. The misting hard hat comprises a hat base having a rigid shell and suspension system, a fluid reservoir attached to the interior of the rigid shell, a misting component for dispersing liquid as a mist, and a distribution system for delivering liquid from the from the reservoir to the misting component under pressure. In one embodiment, the misting component defines a clip on housing that includes a swivel misting nozzle operative to direct mist over a range of directions, thereby provided a large coverage area for mist.

In one embodiment, the hat base may include a plurality of vents for providing ventilation to the reservoir.

It is an object of this invention to provide a hard hat having an integral misting system which operates to cool a wearer by spraying mist on the wearer.

It is another object of this invention to provide a hard hat having an integral misting system which includes a reservoir allow for the storage of liquid to be misted.

It is yet another object of this invention to provide a hard hat having a distribution system which includes a pump and conduit network operative to deliver liquid in the reservoir to a misting duct.

These and other objects will be apparent to one of skill in the art.

### BRIEF DESCRIPTION OF THE DRAWINGS

FIG. 1 is a side elevational view of a hard hat having an integral misting system built in accordance with the present invention.

FIG. 2 is a front elevational view of a hard hat having an integral misting system built in accordance with the present invention.

FIG. 3 is a rear elevational view of a hard hat having an integral misting system built in accordance with the present invention.

FIG. 4 is a side elevational view of a hard hat having an integral misting system built in accordance with the present invention with its hidden internal components shown.

### DETAILED DESCRIPTION OF THE INVENTION

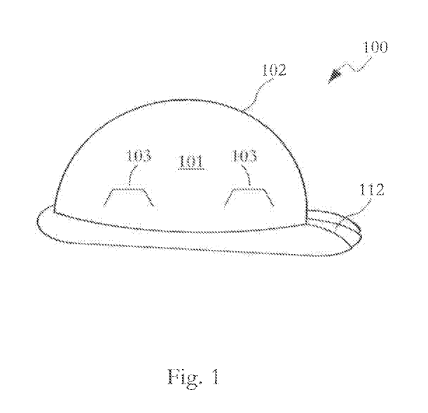

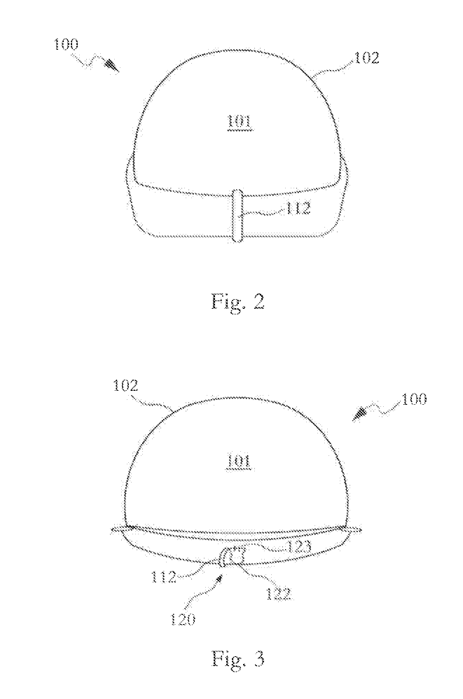

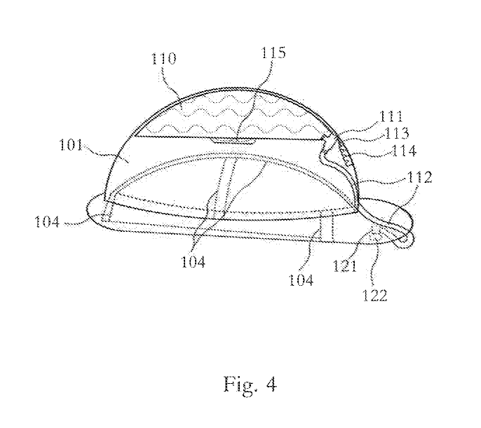

Referring now to the drawings and in particular FIGS. 1, 2, 3, and 4, a misting hard hat 100 is shown having a hat base 101 having a rigid shell 102, a reservoir 110, a distribution system and a misting component 120. In one embodiment, the hat base 101 includes a plurality of vents 103 and a one touch adjustable suspension system inside the shell 102 which includes a plurality of suspension bands 104. It is contemplated, however, that in alternate embodiments, alternate known hard hat designs and a suspension systems may be employed for the hat base 101. For example, in one embodiment, the suspension system includes a headband constructed of a hyper evaporative cooling material or other sweat wicking material.

Disposed in the hat base 101 is a reservoir 110 which can be filled with water (or other liquid) for use by the distribution system and misting component 120. In the illustrated embodiment, the reservoir 110 is positioned inside the hat base 101 against the top of the interior surface of the shell 102. It is contemplated that by positioning the reservoir 110 in this position, it may be held against the shell 102 above where the vents 103 are positioned at the top of the shell 102 and above the suspension bands 104 so that the reservoir is held in a gap space and does not contact a user's head when the misting hard hat 100 is being worn. The vents 103 are positioned lower than the reservoir 110 so that most of the heat from the user's head may be ventilated out of the hat base 101.

The reservoir 110 includes a selectively sealable access sport 115 to allow fluid to be introduced and the sealed therein.

In the preferred embodiment, the reservoir 110 is attached to the hat base 101 though an adhesive or other permanent fastener. In other embodiments, the reservoir 110 may be removably attached through surface fastener, such as a fabric hook and loop fastener.

Also disposed in the hat base 101 is a distribution system which operates to transport water from the reservoir 110 and deliver it to the misting component 120. In one embodiment, the distribution system includes an electric pump 111 which is connected to the reservoir 110, a flexible conduit 112 which is connected to the pump 111 and the misting component 120 and which operates to deliver water pumped from the pump 111 to the misting component 120, an onboard, rechargeable power source 113 which supplies electricity to the pump 111, and a hose clamp 114 which removably connects the conduit 112 to the pump 111.

In one embodiment, the power source 113 defines a battery, such as a lithium ion battery. In one embodiment, the hat base 101 includes a solar panel that is connected to the power source 113 and which generates and supplies electricity to the power source 113.

In one embodiment, the misting component 120 defines a clip on housing 121 that includes a swivel misting nozzle 122, such as the Nozzle Swivel by MISTCOOLING, Inc., which operates to project fluid supplied thereto as a mist. In one embodiment, the housing 121 includes an attached clip 123 to allow it to removably attach to the hat base 101. In other embodiments, the clip may define a discrete body which selectively attaches to both the housing 121 and the hat base 101.

The instant invention has been shown and described herein in what is considered to be the most practical and preferred embodiment. It is recognized, however, that departures may be made therefrom within the scope of the invention and that obvious modifications will occur to a person skilled in the art.

What is claimed is:

1. A misting hard hat, comprising:
a hat base having a rigid shell that includes an interior surface and an exterior surface;
a reservoir integral with the hat base, wherein said reservoir is adapted to hold a supply of a liquid;
a misting component integral with said hat base and adapted to disperse liquid directed thereto as a mist; and
a distribution system integral with said reservoir and said misting component, wherein said distribution system is adapted to receive liquid from the reservoir, pressurize liquid received from the reservoir, and direct pressurized liquid to the misting component.

2. The misting hard hat of claim 1, wherein:
said hat base includes a rear brim; and
said misting component is integral with said rear brim.

3. The misting hard hat of claim 2, wherein said misting component is selectively attachable to the rear brim.

4. The misting hard hat of claim 1, wherein said misting component is selectively attachable to the hat base.

5. The misting hard hat of claim 1, wherein said reservoir is attached to the interior surface of the hat base.

6. The misting hard hat of claim 5, wherein:
said hat base includes a suspension system operative to provide a gap space between the interior surface and the head of a wearer when the hat base is in place on the wearer's head; and
said reservoir is positioned in the gap space on said interior surface such that when the hat base is in place on the wearer's head, the reservoir does not contact the wearer's head.

7. The misting hard hat of claim 1, wherein said misting component includes a swivel misting nozzle.

8. The misting hard hat of claim 1, wherein the distribution system includes an electric pump, a power source, and a flexible conduit.

9. A misting hard hat, comprising:
a hat base having a rigid shell and a rear brim, wherein said hat base includes an interior surface and an exterior surface;
wherein said hat base includes a suspension system operative to provide a gap space between the interior surface and the head of a wearer when the hat base is in place on the wearer's head;
a reservoir attached to the interior surface of the hat base, wherein said reservoir is adapted to hold a supply of water and is positioned in the gap space on said interior surface such that when the hat base is in place on the wearer's head, the reservoir does not contact the wearer's head;
a misting component selectively attachable to the rear brim and adapted to disperse water directed thereto as a mist; and
a distribution system having an electric pump, a power source, and a flexible conduit and integral with said reservoir and said misting component, wherein said distribution system is adapted to receive water from the reservoir, pressurize water received from the reservoir, and direct pressurized water to the misting component.

10. The misting hard hat of claim 10, wherein said misting component includes a swivel misting nozzle.
